(19) United States
(12) Patent Application Publication
(10) Pub. No.: US 2014/0142506 A1
(43) Pub. Date: May 22, 2014

(54) BALLOON CATHETER WITH IMPROVED PUSHABILITY

(71) Applicant: BOSTON SCIENTIFIC SCIMED, INC., Maple Grove, MN (US)

(72) Inventors: KATHERINE M. PRINDLE, ROBBINSDALE, MN (US); PU ZHOU, IRVINE, CA (US); KEN XIAO KANG ZHANG, MAPLE GROVE, MN (US)

(73) Assignee: BOSTON SCIENTIFIC SCIMED, INC., MAPLE GROVE, MN (US)

(21) Appl. No.: 14/081,924

(22) Filed: Nov. 15, 2013

Related U.S. Application Data
(60) Provisional application No. 61/727,491, filed on Nov. 16, 2012.

(51) Int. Cl. A61M 25/10

(52) U.S. Cl. CPC A61M 25/10; A61M 25/1036
USPC 604/103.09; 29/428

(57) ABSTRACT

Embodiments of the disclosure describe a balloon catheter. The balloon catheter may include a catheter shaft including a proximal shaft, a midshaft attached to the proximal shaft, and a distal shaft attached to the midshaft. A balloon may be coupled to the distal shaft. An inflation lumen may be defined in the catheter shaft that extends from the proximal shaft, through the midshaft, and into the distal shaft. The inflation lumen may be in fluid communication with the balloon. A core wire may be extending through a portion of the inflation lumen. A push member may be coupled to the core wire, the push member being configured to engage an inner wall surface of the catheter shaft.

### CROSS-REFERENCE TO RELATED APPLICATIONS

This application claims priority under 35 U.S.C. §119 to U.S. Provisional Application Ser. No. 61/727,491, filed Nov. 16, 2012, the entirety of which is incorporated herein by reference.

### TECHNICAL FIELD

The present invention relates generally to catheters for performing medical procedures. More particularly, the present invention relates to balloon catheters.

### BACKGROUND

A wide variety of intracorporeal medical devices have been developed for medical use, for example, intravascular use. Some of these devices include guidewires, catheters, and the like. These devices are manufactured by any one of a variety of different manufacturing methods and may be used according to any one of a variety of methods. Of the known medical devices and methods, each has certain advantages and disadvantages. There is an ongoing need to provide alternative medical devices as well as alternative methods for manufacturing and using medical devices.

### BRIEF SUMMARY

This disclosure provides design, material, manufacturing method, and use alternatives for medical devices. An example medical device may include a balloon catheter. The balloon catheter may include a catheter shaft including a proximal shaft, a midshaft attached to the proximal shaft, and a distal shaft attached to the midshaft. A balloon may be coupled to the distal shaft. An inflation lumen may be defined in the catheter shaft that extends from the proximal shaft, through the midshaft, and into the distal shaft. The inflation lumen may be in fluid communication with the balloon. A core wire may extend through a portion of the inflation lumen. A push member may be coupled to the core wire, configured to engage an inner wall surface of the catheter shaft.

Another example balloon catheter may include a catheter shaft having a proximal shaft portion, a midshaft portion attached to the proximal shaft portion, and a distal shaft portion attached to the midshaft portion. A balloon may be coupled to the catheter shaft. A guidewire port may be formed in the midshaft portion, the guidewire port being in fluid communication with a guidewire lumen extending along a portion of the catheter shaft. An inflation lumen may be defined in the catheter shaft. The inflation lumen may be in fluid communication with the balloon. A core wire may extend through the inflation lumen. A push member may be attached to the core wire, the push member contacting an inner wall surface of the catheter shaft.

An example method for manufacturing a balloon catheter may include providing a catheter shaft, the catheter shaft including a proximal shaft portion, a midshaft portion attached to the proximal shaft portion, and a distal shaft portion attached to the midshaft portion. A guidewire port may be formed in the midshaft portion, the guidewire port being in fluid communication with a guidewire lumen extending along a portion of the catheter shaft. An inflation lumen may be defined in the catheter shaft that extends from the proximal shaft portion, through the midshaft portion, and into the distal shaft portion. The method may further include providing a core wire having a push member attached thereto. Additionally, the method may include disposing the core wire within the inflation lumen so that the push member contacts an inner wall surface of the catheter shaft.

The above summary of some embodiments is not intended to describe each disclosed embodiment or every implementation of the present disclosure. The Figures, and Detailed Description, which follow, more particularly exemplify these embodiments.

### BRIEF DESCRIPTION OF THE DRAWINGS

The disclosure may be more completely understood in consideration of the following detailed description in connection with the accompanying drawings, in which:

FIG. 1 is a plan view of an example balloon catheter.

FIG. 2 is a cross-sectional view of a portion of the example balloon catheter shown in FIG. 1.

FIG. 3 is a cross-sectional view taken through plane 3-3 in FIG. 2.

FIGS. 4-9 illustrate some of the example method steps for manufacturing the balloon catheter shown in FIGS. 1-3.

FIG. 10 is a side view of an example push member attached to a core wire.

FIG. 11 is a cross-sectional view of a portion of an example balloon catheter having the core wire and the push member as shown in FIG. 10 disposed therein.

FIGS. 12-17 show some of alternative shapes for a push member.

FIG. 18 is a side view of another example push member.

FIG. 19 is a side view of another example push member.

FIG. 20 is a cross-sectional view of the example push member illustrated in FIG. 19.

FIG. 21 is a side view of an example core wire.

FIG. 22 is a partial cross-sectional side view of the example push member shown in FIG. 21 disposed within a catheter shaft.

FIG. 23 is a side view of an example catheter.

While the disclosure is amenable to various modifications and alternative forms, specifics thereof have been shown by way of example in the drawings and will be described in detail. It should be understood, however, that the intention is not to limit the invention to the particular embodiments described. On the contrary, the intention is to cover all modifications, equivalents, and alternatives falling within the spirit and scope of the disclosure.

### DETAILED DESCRIPTION

For the following defined terms, these definitions shall be applied, unless a different definition is given in the claims or elsewhere in this specification.

All numeric values are herein assumed to be modified by the term “about,” whether or not explicitly indicated. The term “about” generally refers to a range of numbers that one of skill in the art would consider equivalent to the recited value (i.e., having the same function or result). In many instances, the terms “about” may include numbers that are rounded to the nearest significant figure.

The recitation of numerical ranges by endpoints includes all numbers within that range (e.g. 1 to 5 includes 1, 1.5, 2, 2.75, 3, 3.80, 4, and 5).

As used in this specification and the appended claims, the singular forms “a”, “an”, and “the” include plural referents unless the content clearly dictates otherwise. As used in this specification and the appended claims, the term “or” is generally employed in its sense including “and/or” unless the content clearly dictates otherwise.

It is noted that references in the specification to “an embodiment”, “some embodiments”, “other embodiments”, etc., indicate that the embodiment described may include one or more particular features, structures, and/or characteristics. However, such recitations do not necessarily mean that all embodiments include the particular features, structures, and/or characteristics. Additionally, when particular features, structures, and/or characteristics are described in connection with one embodiment, it should be understood that such features, structures, and/or characteristics may also be used connection with other embodiments whether or not explicitly described unless clearly stated to the contrary.

The following detailed description should be read with reference to the drawings in which similar elements in different drawings are numbered the same. The drawings, which are not necessarily to scale, depict illustrative embodiments and are not intended to limit the scope of the invention.

Embodiments of the present disclosure may include a medical device for the delivery of diagnostic or therapeutic modalities. The medical device may take the form of a balloon catheter having a catheter shaft with a proximal shaft, mid-shaft attached to the proximal shaft, and a distal shaft attached to the mid-shaft. The balloon catheter may further include a core wire. For adequate and appropriate transmission of push forces, the present disclosure discloses a push member coupled to the core wire. The core wire with push member may be configured to facilitate the transmission of push forces from the proximal shaft to the distal shaft.

Many of the following examples illustrate implementations in which the catheter may be employed to navigate blood vessels. It will be understood that this choice is merely exemplary and the catheter shaft may be used in any desired body location requiring diagnostic or therapeutic modalities without departing from the scope of the present disclosure.

For purposes of this disclosure, “proximal” refers to the end closer to the device operator during use, and “distal” refers to the end further from the device operator during use.

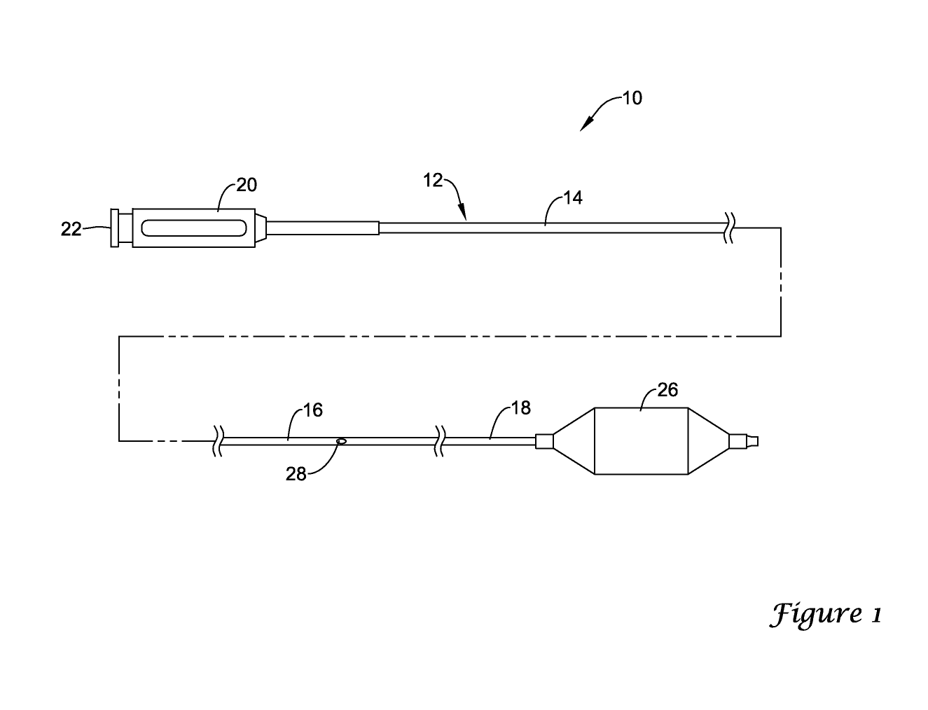

FIG. 1 is a plan view of an example catheter 10, for example a balloon catheter. Balloon catheter may be advanced through blood vessels to treat the desired location. Catheter 10 may include a catheter shaft 12 having a proximal shaft portion 14, a midshaft portion 16 and a distal shaft portion 18. The catheter shaft 12 may be a generally long, flexible tube that may be inserted into the body for a medical diagnosis and/or treatment, for example. The distal shaft portion 18 of the catheter shaft 12 may be softer or more flexible than the proximal shaft portion 14 or midshaft portion 16 so the catheter shaft 12 may more easily navigate inside the patient's body. More particularly, the distal shaft portion 18 may be sufficiently large to allow a therapeutic catheter (e.g. balloon catheter, stent delivery, etc.) to pass through. In some embodiments, proximal shaft portion 14 may be a metallic hypotube. Midshaft portion 16 may be fitted over, fitted within, or abut proximal shaft portion 14, as appropriate. Likewise, distal shaft portion 18 may be fitted over, fitted within, or abut the midshaft portion 16. These are examples, as any suitable arrangement may be utilized. A hub 20 may be attached to proximal shaft portion 14. Hub 20 may include one or more ports such as, for example, a port 22.

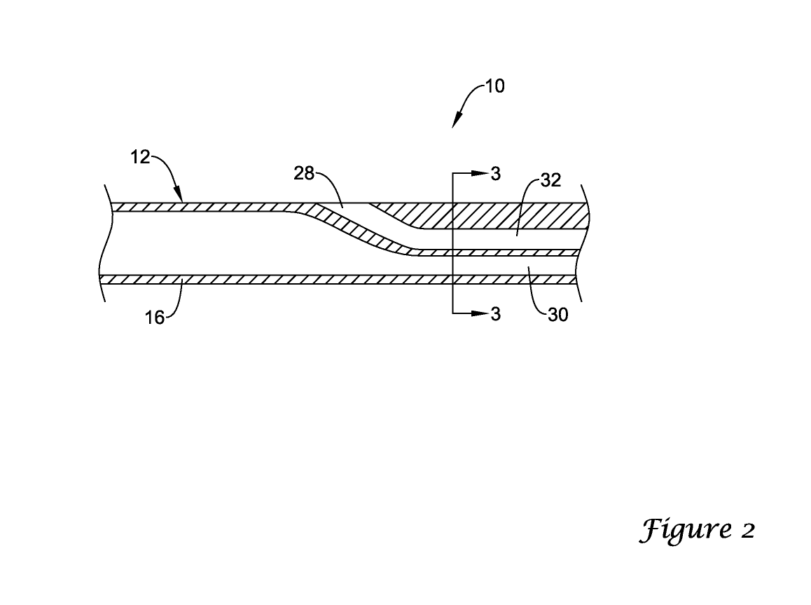

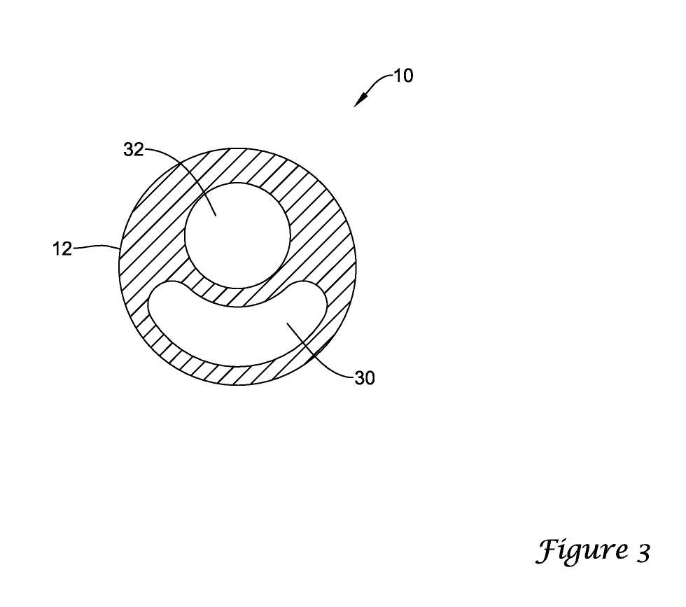

An expandable balloon 26 may be attached to distal shaft portion 18. Balloon 26 may be expanded by infusing inflation media through an inflation lumen 30, which is shown in FIG. 2. In at least some embodiments, port 22 may provide access to inflation lumen 30. Accordingly, a suitable inflation device may be attached to port 22 and inflation media may be passed through inflation lumen 30 to inflate balloon 26. Along a region of midshaft portion 16, inflation lumen 30 may have an annular shape as seen in FIG. 3. This may be due to the formation of a guidewire port 28 in midshaft portion 16. Guidewire lumen 32 may have a circular shape. Some additional details regarding the formation of guidewire port 28 and/or inflation lumen 30 are provided herein.

As indicated above, guidewire port 28 may be formed in midshaft portion 16. Guidewire port 28 may be known as port joint. At this port 28, distal shaft portion 18 may come together with proximal shaft portion 14. In other words, midshaft portion 16 may facilitate bond between the proximal shaft portion 14 and distal shaft portion 18. For example, guidewire port 28 may be an opening extending through the wall of midshaft portion 16 that provides access to a guidewire lumen 32. In the embodiment depicted in FIG. 2, guidewire port 28 is positioned at a location that is distal to the proximal end of catheter shaft 12. When so arranged, catheter 10 may be a single-operator-exchange or rapid-exchange catheter, which allows catheter 10 to be used with a shorter guidewire. As such, guidewire lumen 32 may extend over only a portion of the length of catheter shaft 12. For example, guidewire lumen 32 may extend along distal shaft portion 18 and part of midshaft portion 16. Other embodiments, however, are contemplated where catheter 10 is an over-the-wire catheter or fixed wire catheter. In these embodiments, guidewire lumen 32 may extend along essentially the entire length of catheter shaft 12.

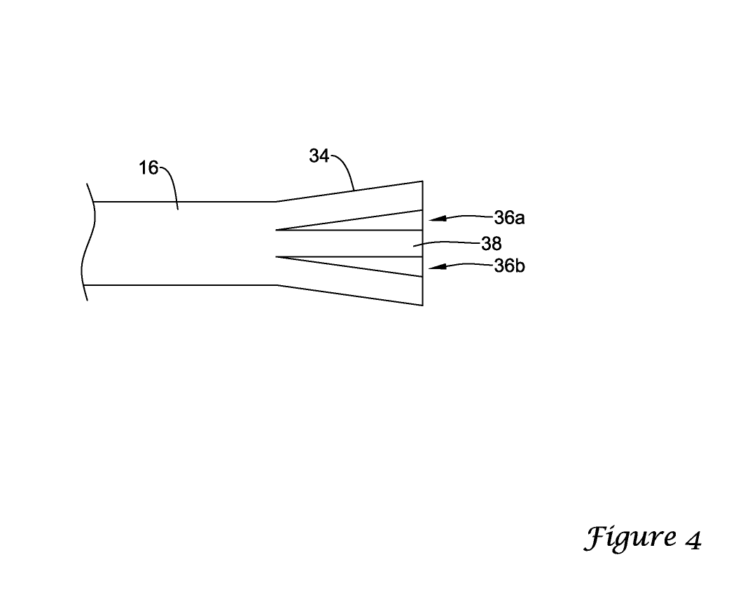

FIGS. 4-9 illustrate some of the processing steps that may be utilized to form catheter 10 and/or catheter shaft 12. For example, FIG. 4 shows part of midshaft portion 16. Here it can be seen that a distal end 34 of midshaft portion 16 may be flared or otherwise enlarged. In addition, one or more cuts or slots, for example cuts 36a/36b, may be formed in distal end 34 of midshaft portion 16. A tongue 38 may be defined between cuts 36a/36b.

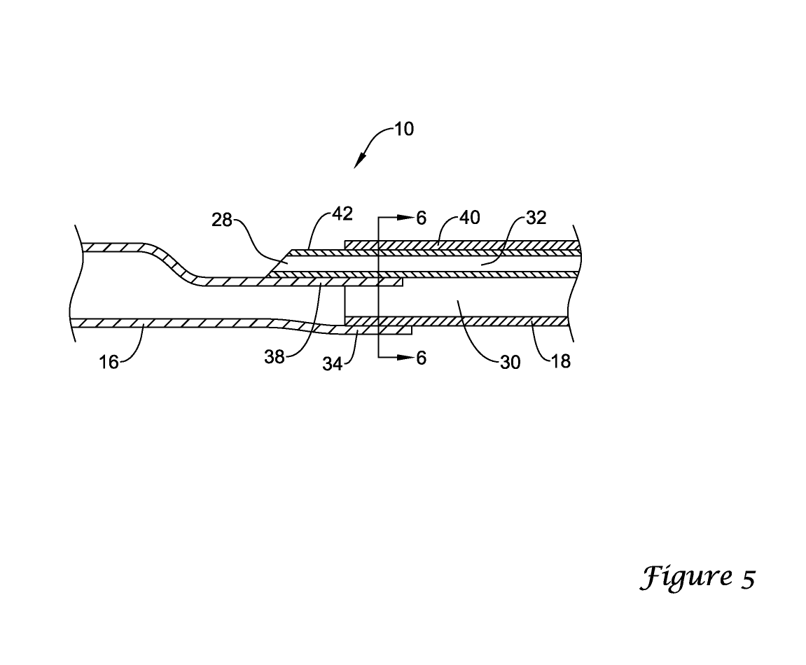

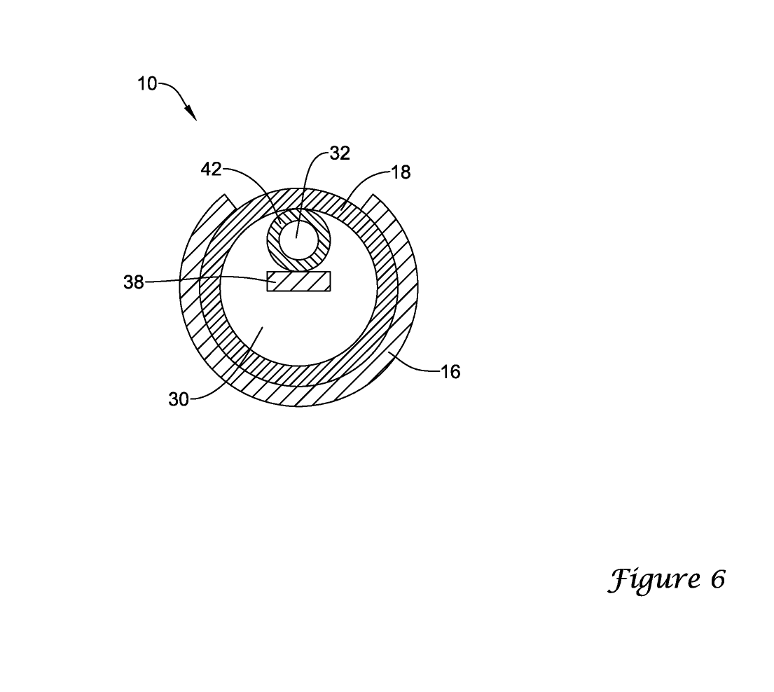

A proximal end 40 of distal shaft portion 18 may be disposed within the enlarged distal end 34 of midshaft portion 16 as shown in FIG. 5. In doing so, tongue 38 may be pressed inward and form a shelf or ledge. This may create or define a guidewire ramp in catheter shaft 12 adjacent to guidewire port 28. In particular, the guidewire ramp may be defined for the portion of the tube 42 that direct the guidewire up to that port 28. A distal inner tube 42 may be disposed within distal shaft portion 18 and may rest upon the ledge formed by tongue 38. Distal inner tube 42 may ultimately form guidewire lumen 32 as described in more detail below. The arrangement of distal inner tube 42 relative to tongue 38, midshaft portion 16, and distal shaft portion 18 can also be seen in FIG. 6.

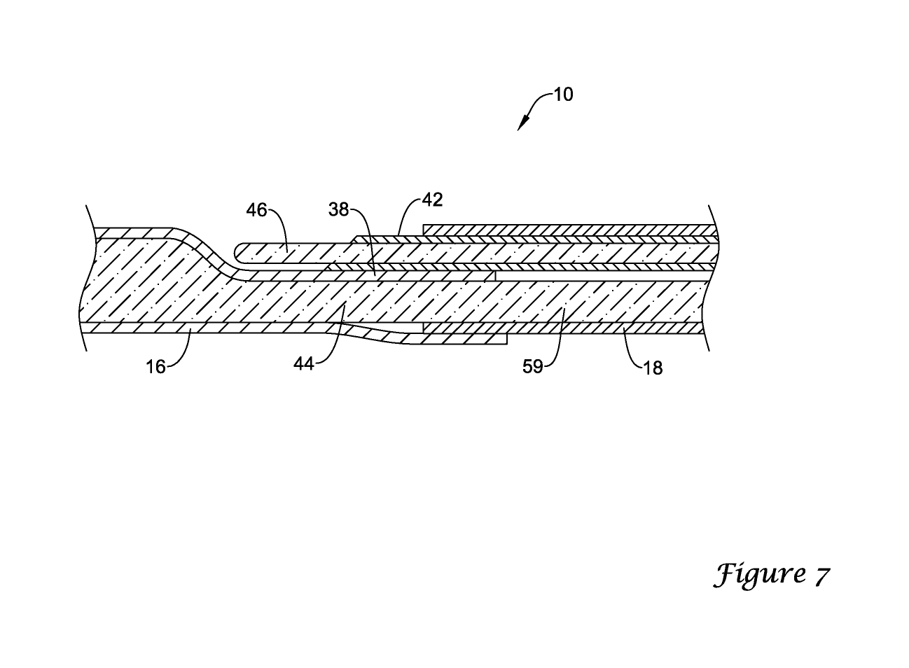

When suitably arranged, a first mandrel 44 may be inserted within a portion of distal shaft portion 18 and midshaft portion 16 as shown in FIG. 7. Mandrel 44 may include a distal section 59. Likewise, a second mandrel 46 may be inserted within distal inner tube 42. Mandrels 44/46 are generally configured to maintain lumens 30/32 when catheter shaft 12 is subjected to heat and/or further processing as described in more detail below.

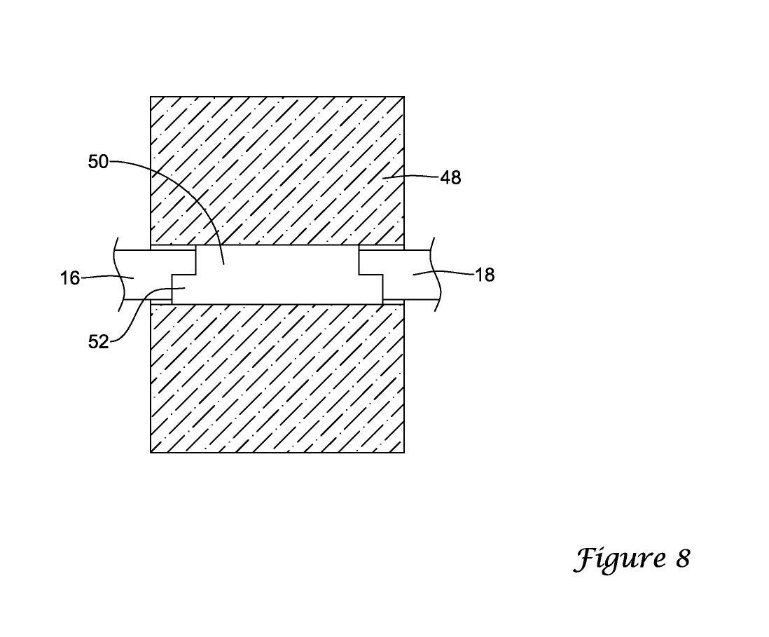

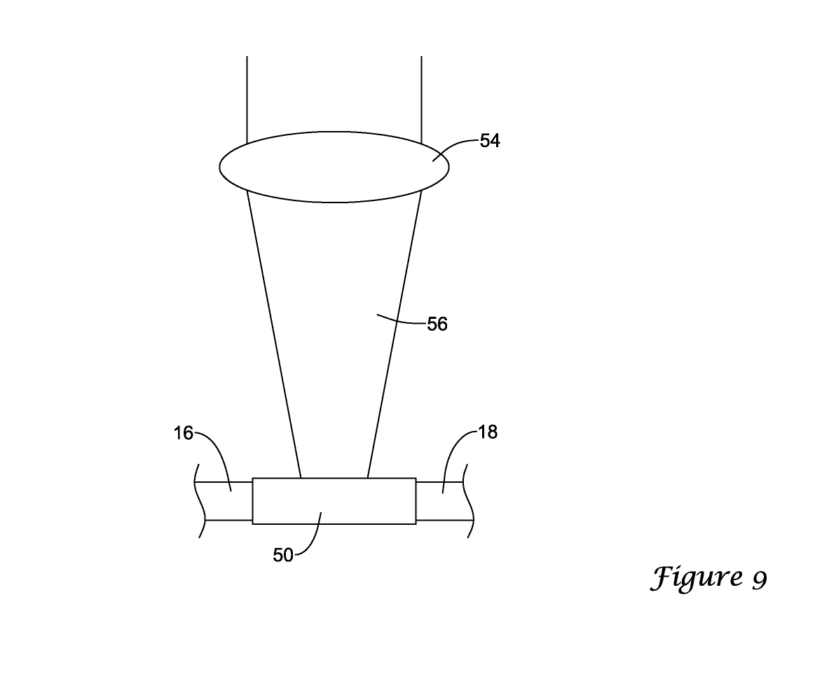

With mandrels 44/46 in place, midshaft portion 16 and distal shaft portion 18 may be disposed within a compression fixture 48 as shown in FIG. 8. A sleeve 50 may be disposed over a region of midshaft portion 16 and distal shaft portion 18. Sleeve 50 may include one or more flanking ears 52, which may aid in removal of sleeve 50 upon completion of the manufacturing process. Finally, heat may be applied to sleeve 50. This may include the use of a lens 54 to focus heat (e.g., laser energy 56) onto sleeve 50 as depicted in FIG. 9. When heated, midshaft portion 16, distal shaft portion 18, and distal inner tube 42 may melt together. Mandrels 44/46 can be removed, thereby defining inflation lumen 30 and guidewire lumen 32, respectively, and the result may be the formation of catheter shaft 12 as shown herein.

Catheters such as catheter 10 may be designed to have increased or increasing distal flexibility. This may be desirable because portions of the catheter 10, particularly distal portions, may need to navigate sharp bends or turns within the vasculature. Because of the relatively high level of flexibility in some catheters, it may be challenging to push the catheter through the vasculature in a reliable manner. In other words, increased distal flexibility, while being desirable for allowing the catheter to navigate the tortuous anatomy, may make it more difficult to “push” the catheter through the anatomy.

Existing medical devices may use core wires for providing transitional support and kink resistance. In some designs, the core wire may be generally freely disposed within the inflation lumen. In such designs, the core wire may not contribute to the pushability of the catheter. As set out in present disclosure, however, the core wire is presented that may be used to improve pushability of the catheter without sacrificing other performance characteristics of the balloon catheter.

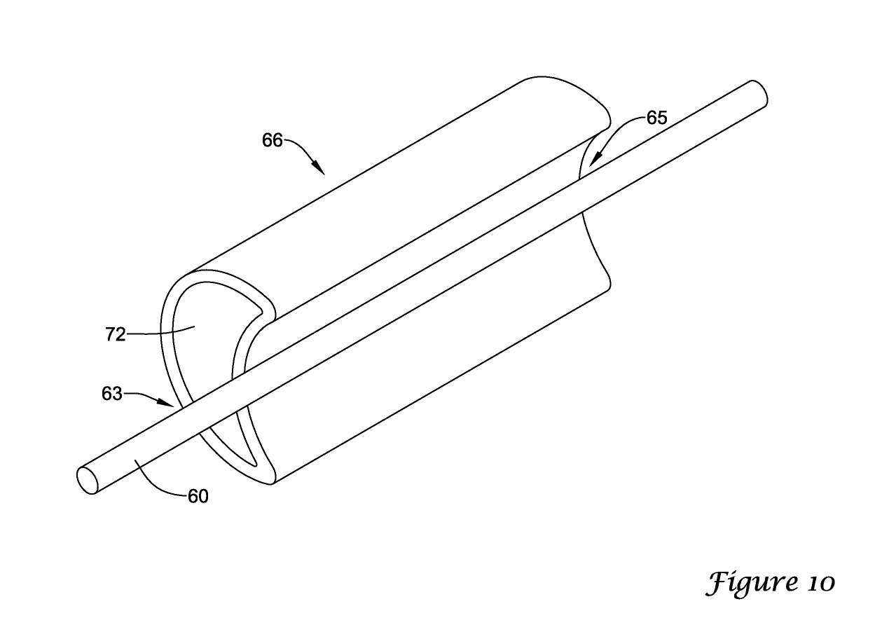

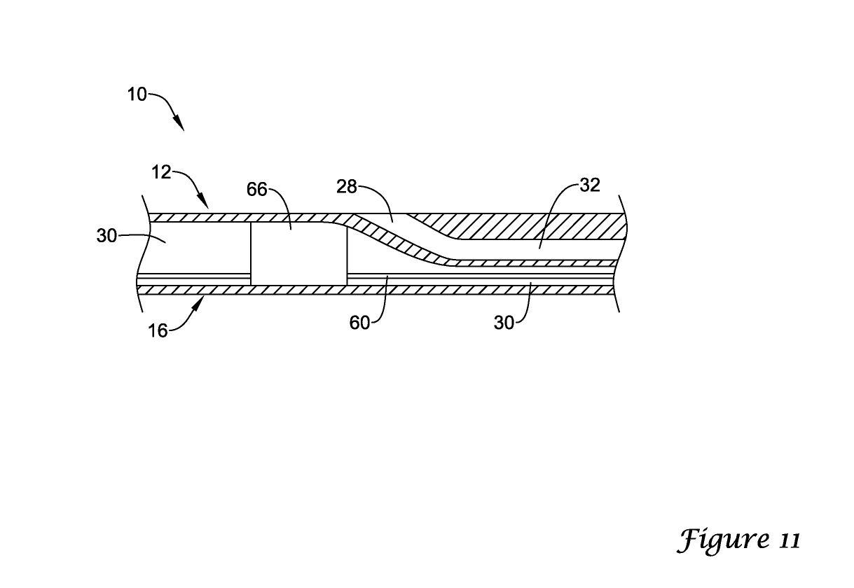

FIGS. 10 and 11 illustrate portions of balloon catheter 10, which include design features that may improve the pushability of catheter 10. For example, FIG. 10 illustrates a core wire 60 with a push member 66 coupled thereto. Push member 66 is generally shaped and sized to fit within inflation lumen 30 as shown in FIG. 11. Thus, when push member 66 approaches the necked-down portion of inflation lumen 30, it makes contact or otherwise engages an inner wall of catheter shaft 12. Accordingly, force applied by the operator to catheter shaft 12 (and/or core wire 60) may be transmitted along the length of catheter shaft 12 and through push member 66 to distal portions of the catheter shaft 12 and/or catheter shaft 12 as a whole. In that manner, the catheter shaft 12 can much more easily be pushed through obstructions or into a tortuous portion of the patient's vasculature. For example, the inclusion of push member 66 may increase the pushability of catheter 10 by about 5-100% or more, about 10-80% or more, about 20-60% or more, or about 30-50% or more. These are just examples.

In some embodiments, push member 66 may be bonded to the inner wall surface of the catheter shaft 12. In other embodiments, push member 66 may be wedged into inflation lumen 30 to secure push member 66. In still other embodiments, core wire 60 and push member 66 may be slidable within catheter shaft 12. This may allow core wire 60 and push member 66 to be slid into catheter shaft 12 (e.g., within inflation lumen 30) when additional transmission of push forces is desired and allow core wire 60 and push member 66 to be proximally retracted or removed from catheter shaft 12 when additional transmission of push forces is no longer needed.

The push member 66 may be an elongate tubular structure made of a suitable material such as a polymer or metal to provide longitudinal stiffness. The shape and thickness of push member 66 may be varied to provide a desired pushing force. In at least some embodiments, push member 66 is at least partially tubular and defines a lumen 72. In some embodiments, multiple lumens may be employed. Lumen 72 of push member 66 may extend longitudinally through the push member 66. This lumen 72 allows inflation fluid to flow through inflation lumen 30 (and through lumen 72) with minimal disruption in the flow of inflation media. As such, inflation and deflation of balloon 26 can occur with little or no disruption. For example, the inflation and deflation time of balloon 26 (in catheters including push member 66) may be substantially the same as for similar balloons (in catheter lacking push member 66).

A number of structural variations may be envisioned and incorporated by those of skill in the art. In some embodiments, for example, push member 66 may be formed of a relatively flexible material and provided with a mechanism for manual expansion by pumping air, saline, or other suitable inflation fluid. The expanded push member 66 could then transfer pushing forces.

Core wire 60 may extend from at least from the proximal end of the catheter 10 (not shown) to guidewire port 28, and to a position distal of guidewire port 28. This may include core wire 60 extending the length of the catheter shaft 12. Core wire 60 may generally take the form of a singular wire or rod having a solid cross section. In other embodiments, core wire 60 may be tubular or include portions that are tubular and thus, hollow. In still other embodiments, core wire 60 may include a plurality of wire filaments that may be longitudinally aligned, twisted, braided, or the like. Further, core wire 60 may have a substantially uniform diameter or dimension throughout the length, or it may have a non-uniform cross-section in some implementations. For example, a tapering region may allow for a gradual transition in flexibility along portions of the length of catheter shaft 12 (e.g., at or near transitions between portions 14/16/18).

The core wire 60 may be attached to the body of the push member 66 at the edges 63, 65 of push member, or otherwise. Attachment may be made through any other suitable method such as an adhesive bond, thermal bond, or the like. Welding, brazing, or other conventional joining techniques can be employed as desired. Alternatively, core wire 60 may run through the body or wall of push member 66, or though lumen 72. In such arrangements, core wire 60 could be attached to push member 66 at any convenient point or surface.

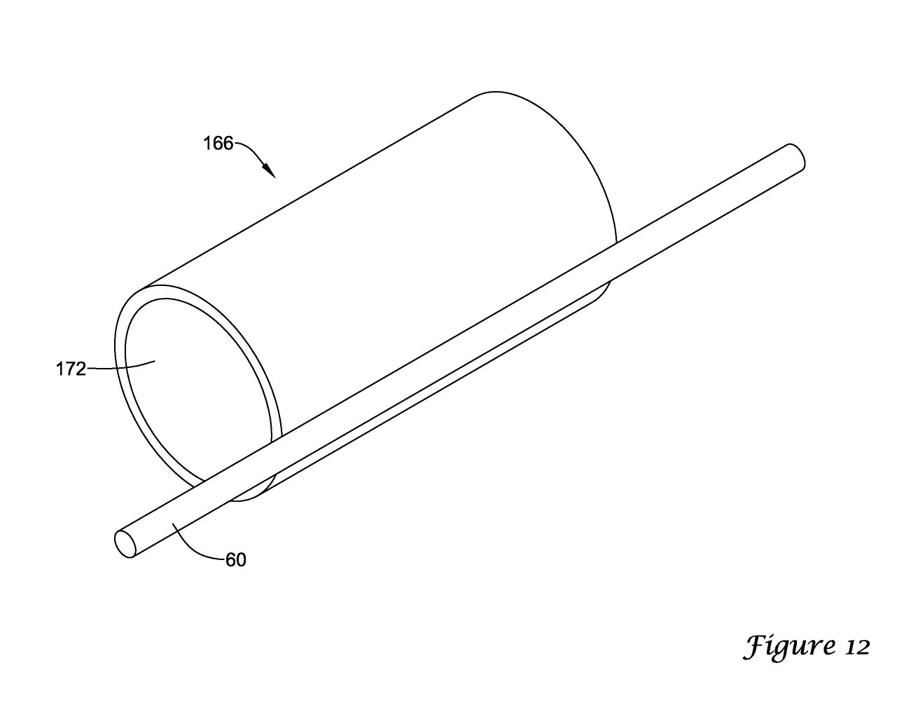

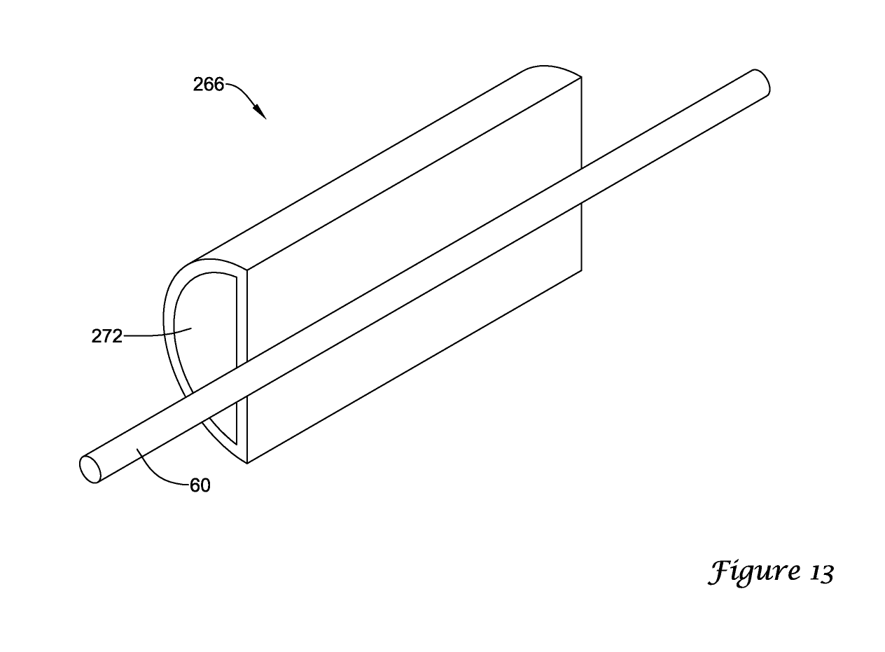

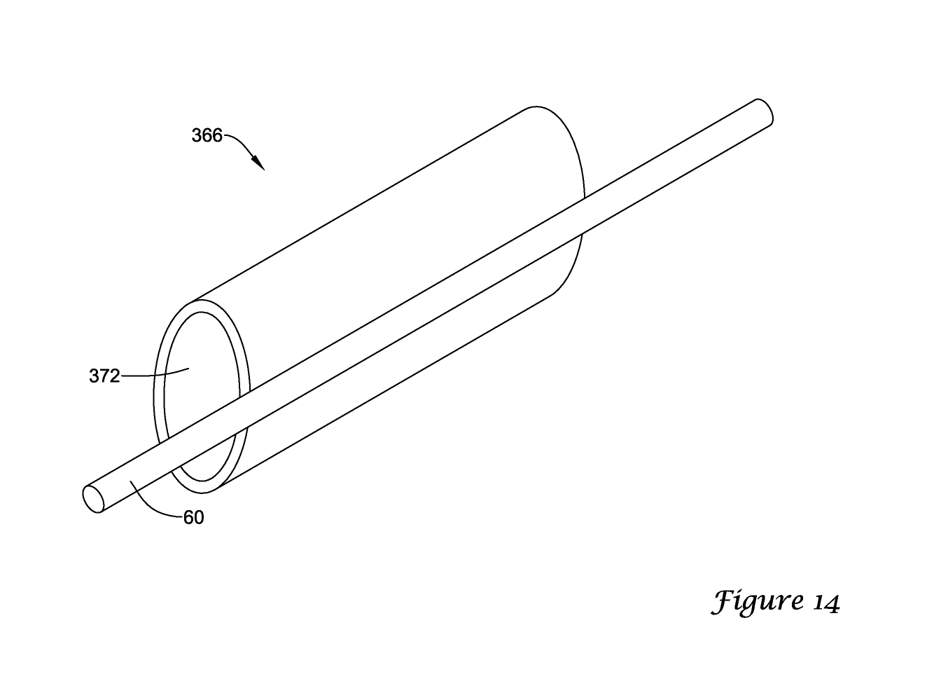

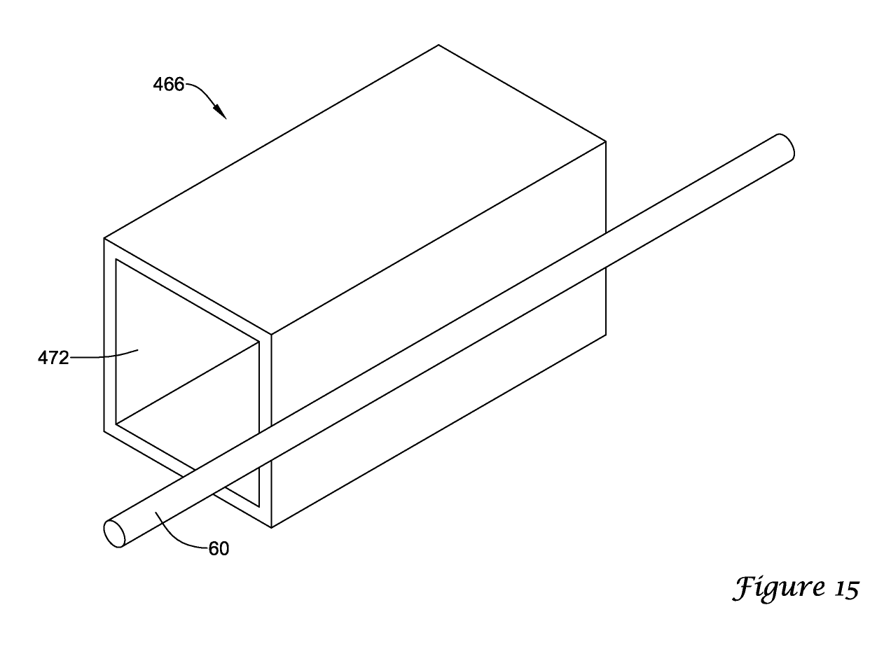

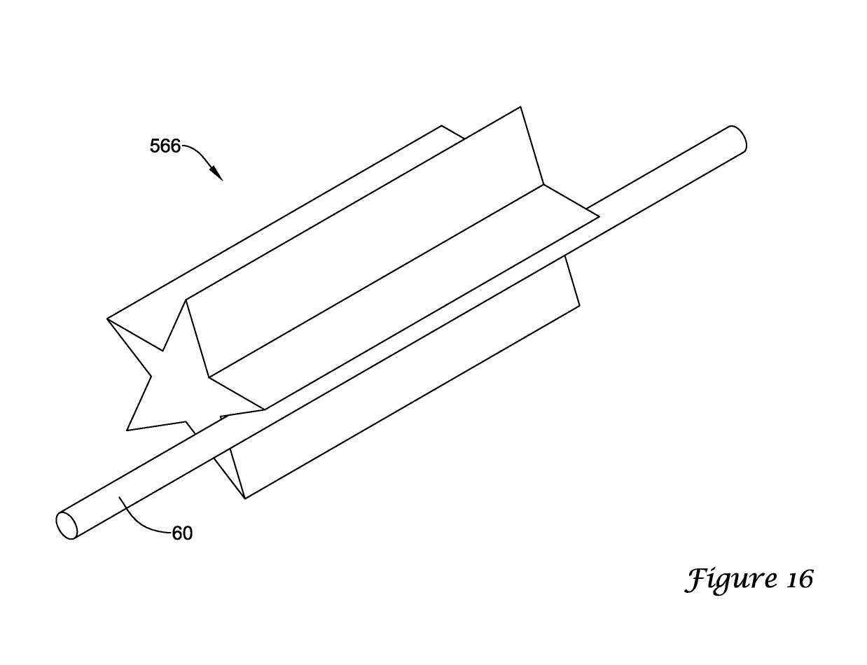

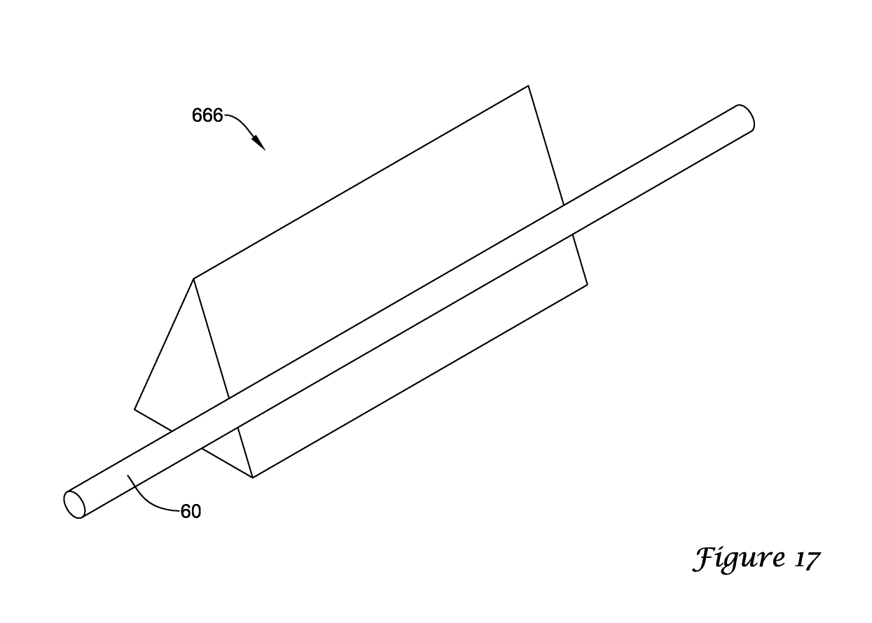

The embodiment of FIG. 10 shows push member 66 having a crescent-shaped cross-section. As noted above, push member 66 may conveniently be shaped to fit the proximal portion of inflation lumen 30, and thus a variety of shapes could be employed. Other cross-sectional shapes may include oval, semi-circular, cylindrical, square, star, triangle, as depicted in FIGS. 11-17. In particular, FIG. 12 depicts a generally cylindrical push member 166 with lumen 172. FIG. 13 illustrates a semi-circular push member 266 with 272. FIG. 14 illustrates an oval push member 366 having lumen 372. FIG. 15 further shows an embodiment where push member 466 may have square cross-section and lumen 472. In the discussed embodiments, push member 66/166/266/366/466 may have tubular structure and may further have lumens defined therein for passing inflation fluid. But in some embodiments, such as those shown in FIGS. 16-17, a non-tubular or solid push member may be utilized. For example, in one embodiment shown in FIG. 16, push member 566 may have a star-shaped cross-section, and FIG. 17 shows that push member 666 may be of triangular shape. Such non-tubular structures may lack a lumen, and for such designs, provision must be made for the flow of fluid through inflation lumen 30. In either of the illustrated embodiments, for example, the illustrated push members could be deployed in a circular inflation lumen 30 (not shown), which would allow fluid flow around push member 66. These are just examples. Other shapes and configurations are contemplated for push members.

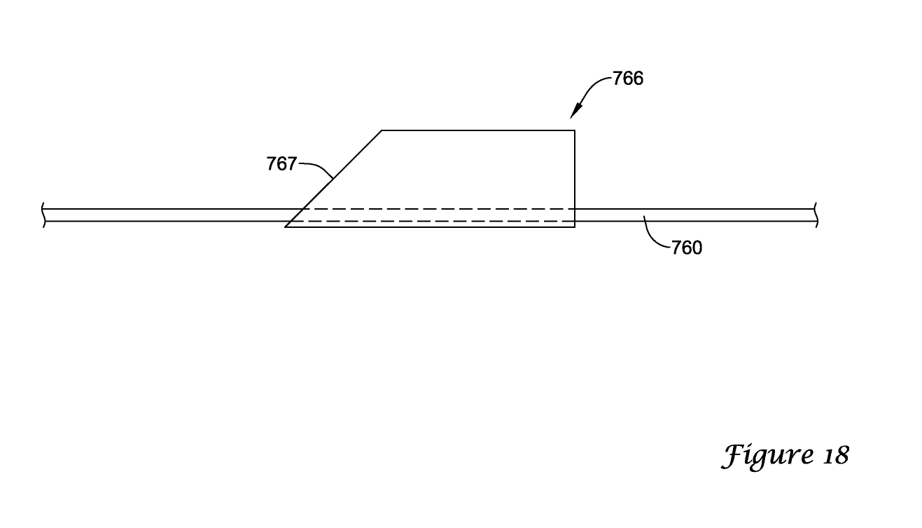

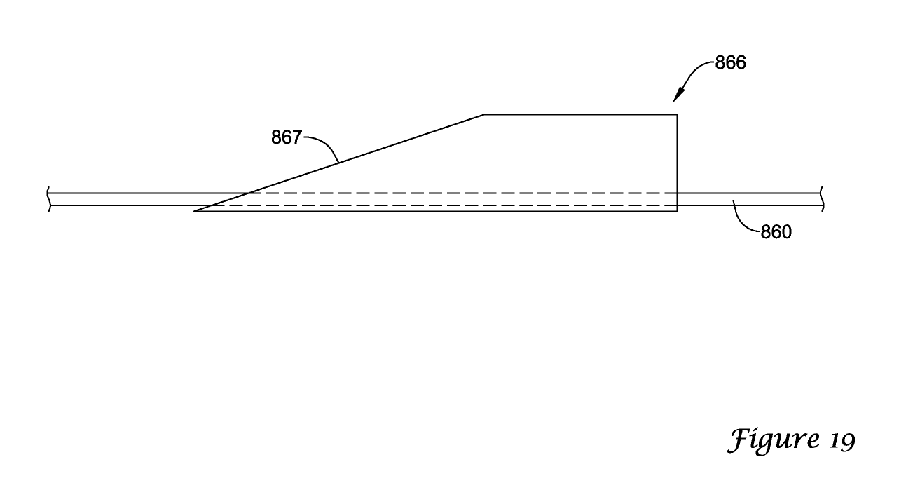

FIG. 18 illustrates another example push member 766 coupled with or otherwise secured to core wire 760. In this example, push member 766 takes the form of a generally rounded or cylindrical body with an angled end 767. Similarly, FIG. 19 illustrates push member 866 secured to core wire 860, which also may take the form of a generally rounded or cylindrical body with an angled end 867. Push members 766/866 may be used with any of the catheters and/or catheter shafts disclosed herein.

Push members 766/866 may vary in form. In some examples, push members 766/866 may have an inner diameter of about 0.02-0.04 inches (e.g., about 0.028 inches) and an outer diameter of about 0.025-0.045 inches (e.g., about 0.034 inches). The length of push members 766/866 may be about 1-10 mm (e.g., about 3 mm and 5 mm, respectively, for push member 766 and 866). The orientation of angled end 767/867 may be oriented at a relatively “steep” angle (e.g., over a length of about 1 mm in push member 766) or at a relatively “gradual” angle (e.g., over a length of about 3 mm in push member 866). These dimensions and configurations are just examples.

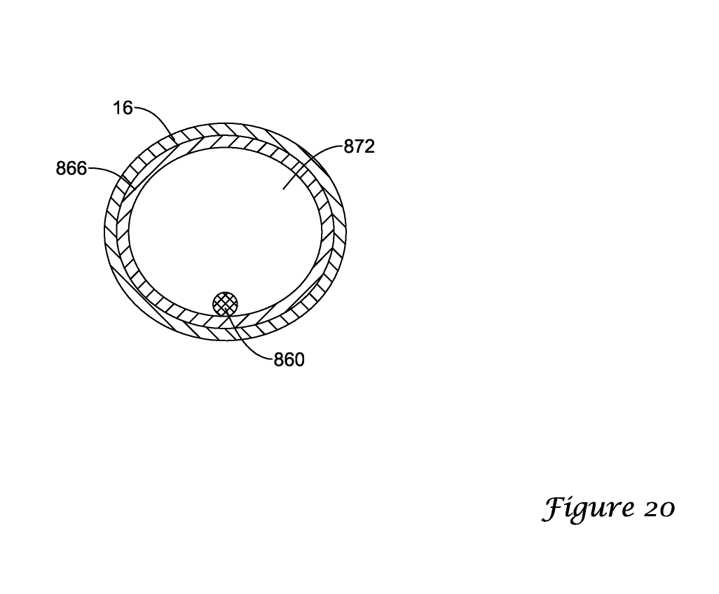

In at least some embodiments, core wire 860 may be disposed along and bonded to (e.g., using an adhesive bond, thermal bond, or the like) the inner surface of push member 866 as shown in FIG. 20. The same configuration may also be utilized for push member 766. Alternative bonds may also be utilized. When used with a catheter shaft, push member 766/866 may be disposed within midshaft portion 16. For example, push member 766/866 may be disposed within inflation lumen 30 so that angled end 767/867 may engage an inner wall surface of catheter shaft 12. This may allow push member 766/866 to aid in the transmission of force along the length of catheter shaft 12 in a manner similar to other push members disclosed hererin.

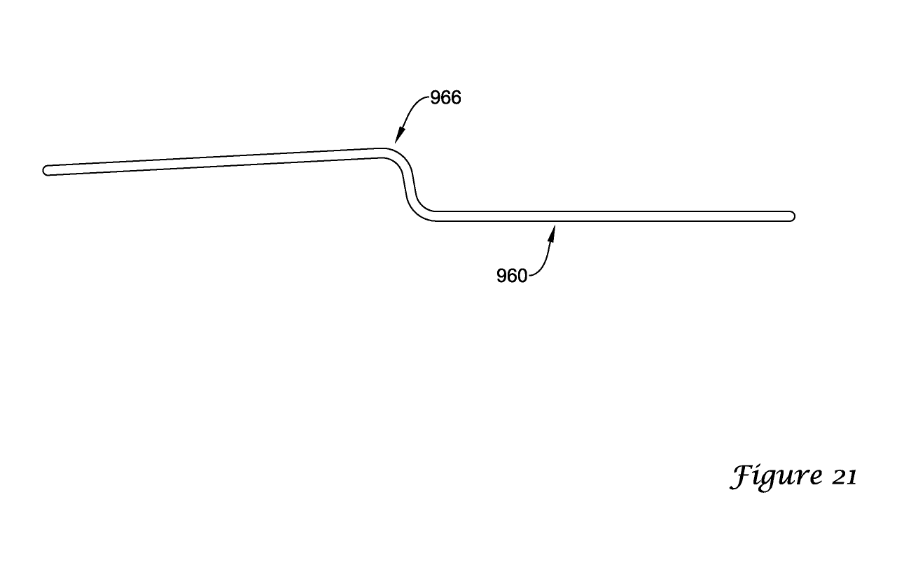

FIG. 21 illustrates another example core wire 960 having a bent or “zig-zag” push member region 966. The form and/or configuration of core wire 960 may vary. For example, the angle or shape of the bend in push member region 966 may vary. In general, the shape of push member region 966 may be configured so that core wire 960 may be used to aid in the transmission of forces along a catheter shaft (e.g., as discussed herein). Core wire 960 may have an outer diameter of about 0.05-0.15 inches (e.g. about 0.08 inches) and the length of push member region 966 may be about 0.5-3 mm (e.g., about 1 mm). These are just examples. Other dimensions and/or configurations are contemplated.

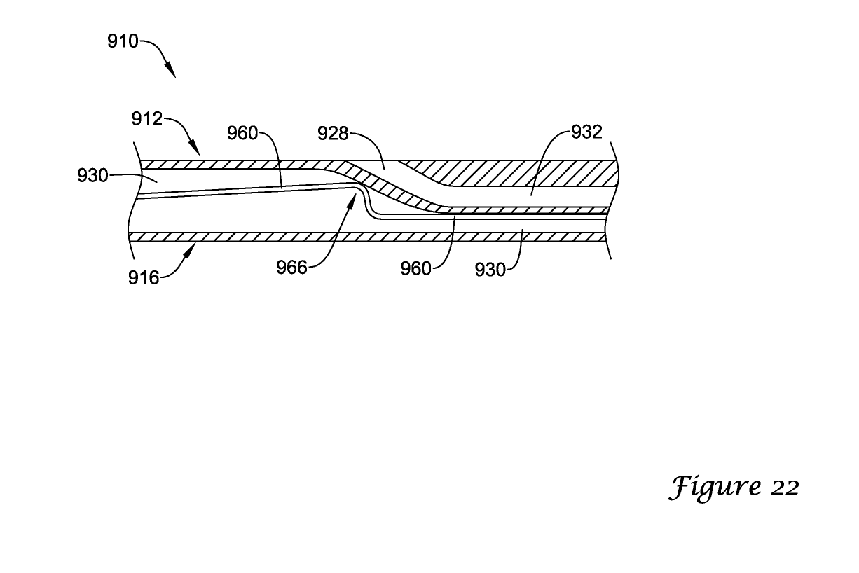

FIG. 22 illustrates catheter 910 including core wire 960. Catheter 910, which may be similar in form and function to other catheters disclosed herein, may include catheter shaft 912 having midshaft region 916, guidewire port 928, guidewire lumen 932, and inflation lumen 930. Core wire 960 may be disposed within inflation lumen 930 and oriented so that push member region 966 may engage an inner wall surface of catheter shaft 912. This may allow push member region 966 to aid in the transmission of force along the length of catheter shaft 912 in a manner similar to other push members disclosed hererin.

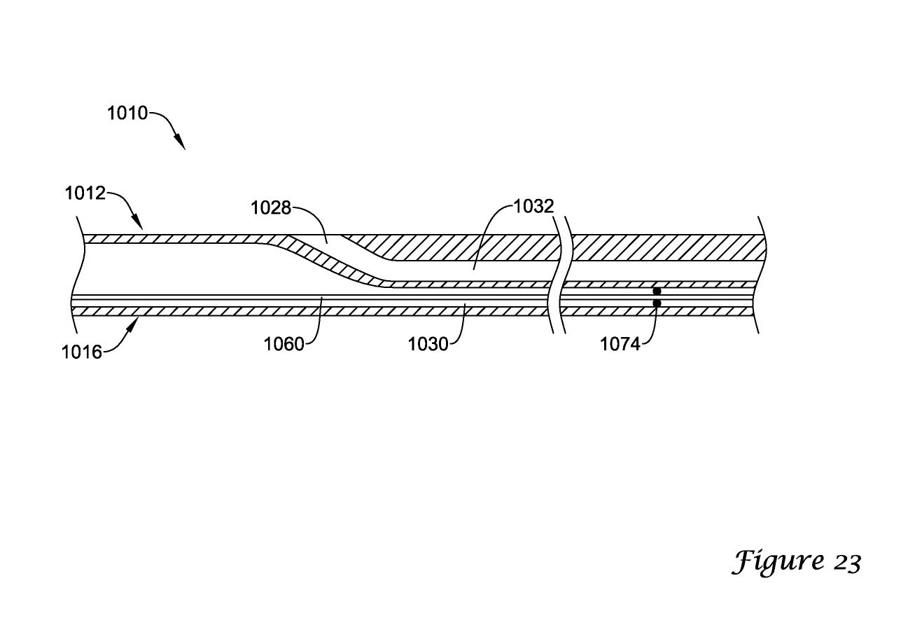

FIG. 23 is another example catheter 1010 that may be similar in form and function to other catheters disclosed herein. Here, a bond member 1074 is used to secure core wire 1060 to an inner wall surface of catheter shaft 1012 (e.g., within inflation lumen 1030). Bond member 1074 may vary in form. For example, bond member 1074 may include an adhesive bond, a thermal bond, or the like. Other types of bonds are contemplated. The bonding or fusing method may largely depend on the material of the catheter shaft 1012, welding or brazing being usable for a metallic member but chemical means being preferred for polymer materials. The bond member 1074 may be disposed distally of guidewire port 1028. In this embodiment, guidewire lumen may be shown as 1032 and midshaft portion by 1016.

Here, a pushing force applied to the proximal portion of core wire 1060 can be transferred to a more distal position on catheter shaft 1012. For example, the inclusion of bond member 1074 may increase the pushability of catheter 1010 by about 1-50% or more, about 5-30% or more, or about 10-20% or more. These are just examples.

In addition, the illustrated embodiment offers the possibility of lengthening the longitudinal extent of bond member 1074. A longer bond member 1074 may have the effect of reducing the force per unit area being transferred from core wire 1060 to catheter shaft 1012, which in turn may increase the amount of force that can be transferred. Additionally, a longer bond member 1074 length could reduce any propensity to kink.

The materials that can be used for the various components of catheter 10 (and/or other catheters disclosed herein) may include those commonly associated with medical devices. For simplicity purposes, the following discussion makes reference to catheter shaft 12 and other components of catheter 10. However, this is not intended to limit the devices and methods described herein, as the discussion may be applied to other similar tubular members and/or components of tubular members or devices disclosed herein.

Catheter shaft 12 and/or other components of catheter 10 may be made from a metal, metal alloy, polymer (some examples of which are disclosed below), a metal-polymer composite, ceramics, combinations thereof, and the like, or other suitable material. Some examples of suitable polymers may include polytetrafluoroethylene (PTFE), ethylene tetrafluoroethylene (ETFE), fluorinated ethylene propylene (FEP), polyoxymethylene (POM, for example, DELRIN® available from DuPont), polyether block ester, polyurethane (for example, Polyurethane 85A), polypropylene (PP), polyvinylchloride (PVC), polyether-ester (for example, ARNITEL® available from DSM Engineering Plastics), ether or ester based copolymers (for example, butylene/poly(alkylene ether) phthalate and/or other polyester elastomers such as HYTREL® available from DuPont), polyamide (for example, DURETHAN® available from Bayer or CRISTAMID® available from Elf Atochem), elastomeric polyamides, block polyamide/ethers, polyether block amide (PEBA, for example available under the trade name PEBAX®), ethylene vinyl acetate copolymers (EVA), silicones, polyethylene (PE), Marlex high-density polyethylene, Marlex low-density polyethylene, linear low density polyethylene (for example REXELL®), polyester, polybutylene terephthalate (PBT), polyethylene terephthalate (PET), polytrimethylene terephthalate, polyethylene naphthalate (PEN), polyetheretherketone (PEEK), polyimide (PI), polyetherimide (PEI), polyphenylene sulfide (PPS), polyphenylene oxide (PPO), poly paraphenylene terephthalamide (for example, KEVLAR®), polysulfone, nylon, nylon-12 (such as GRILAMID® available from EMS American Grilon), perfluoro(propyl vinyl ether) (PFA), ethylene vinyl alcohol, polyolefin, polystyrene, epoxy, polyvinylidene chloride (PVdC), poly(styrene-b-isobutylene-b-styrene) (for example, SIBS and/or SIBS 50A), polycarbonates, ionomers, biocompatible polymers, other suitable materials, or mixtures, combinations, copolymers thereof, polymer/metal composites, and the like. In some embodiments the sheath can be blended with a liquid crystal polymer (LCP). For example, the mixture can contain up to about 6 percent LCP.

Some examples of suitable metals and metal alloys include stainless steel, such as 304V, 304L, and 316LV stainless steel; mild steel; nickel-titanium alloy such as linear-elastic and/or super-elastic nitinol; other nickel alloys such as nickel-chromium-molybdenum alloys (e.g., UNS: N06625 such as INCONEL® 625, UNS: N06022 such as HASTELLOY® C-22®, UNS: N10276 such as HASTELLOY® C276®, other HASTELLOY® alloys, and the like), nickel-copper alloys (e.g., UNS: N04400 such as MONEL® 400, NICKELVAC® 400, NICORROS® 400, and the like), nickel-cobalt-chromium-molybdenum alloys (e.g., UNS: R30035 such as MP35-N® and the like), nickel-molybdenum alloys (e.g., UNS: N10665 such as HASTELLOY® ALLOY B2®), other nickel-chromium alloys, other nickel-molybdenum alloys, other nickel-cobalt alloys, other nickel-iron alloys, other nickel-copper alloys, other nickel-tungsten or tungsten alloys, and the like; cobalt-chromium alloys; cobalt-chromium-molybdenum alloys (e.g., UNS: R30003 such as ELGILOY®, PHYNOX®, and the like); platinum enriched stainless steel; titanium; combinations thereof; and the like; or any other suitable material.

As alluded to herein, within the family of commercially available nickel-titanium or nitinol alloys, is a category designated “linear elastic” or “non-super-elastic” which, although may be similar in chemistry to conventional shape memory and super elastic varieties, may exhibit distinct and useful mechanical properties. Linear elastic and/or non-super-elastic nitinol may be distinguished from super elastic nitinol in that the linear elastic and/or non-super-elastic nitinol does not display a substantial “superelastic plateau” or “flag region” in its stress/strain curve like super elastic nitinol does. Instead, in the linear elastic and/or non-super-elastic nitinol, as recoverable strain increases, the stress continues to increase in a substantially linear, or a somewhat, but not necessarily entirely linear relationship until plastic deformation begins or at least in a relationship that is more linear that the super elastic plateau and/or flag region that may be seen with super elastic nitinol. Thus, for the purposes of this disclosure linear elastic and/or non-super-elastic nitinol may also be termed “substantially” linear elastic and/or non-super-elastic nitinol.

In some cases, linear elastic and/or non-super-elastic nitinol may also be distinguishable from super elastic nitinol in that linear elastic and/or non-super-elastic nitinol may accept up to about 2-5% strain while remaining substantially elastic (e.g., before plastically deforming) whereas super elastic nitinol may accept up to about 8% strain before plastically deforming. Both of these materials can be distinguished from other linear elastic materials such as stainless steel (that can also can be distinguished based on its composition), which may accept only about 0.2 to 0.44 percent strain before plastically deforming.

In some embodiments, the linear elastic and/or non-super-elastic nickel-titanium alloy is an alloy that does not show any martensite/austenite phase changes that are detectable by differential scanning calorimetry (DSC) and dynamic metal thermal analysis (DMTA) analysis over a large temperature range. For example, in some embodiments, there may be no martensite/austenite phase changes detectable by DSC and DMTA analysis in the range of about −60 degrees Celsius (° C.) to about 120° C. in the linear elastic and/or non-super-elastic nickel-titanium alloy. The mechanical bending properties of such material may therefore be generally inert to the effect of temperature over this very broad range of temperature. In some embodiments, the mechanical bending properties of the linear elastic and/or non-super-elastic nickel-titanium alloy at ambient or room temperature are substantially the same as the mechanical properties at body temperature, for example, in that they do not display a super-elastic plateau and/or flag region. In other words, across a broad temperature range, the linear elastic and/or non-super-elastic nickel-titanium alloy maintains its linear elastic and/or non-super-elastic characteristics and/or properties.

In some embodiments, the linear elastic and/or non-super-elastic nickel-titanium alloy may be in the range of about 50 to about 60 weight percent nickel, with the remainder being essentially titanium. In some embodiments, the composition is in the range of about 54 to about 57 weight percent nickel. One example of a suitable nickel-titanium alloy is FHP-NT alloy commercially available from Furukawa Techno Material Co. of Kanagawa, Japan. Some examples of nickel titanium alloys are disclosed in U.S. Pat. Nos. 5,238,004 and 6,508,803, which are incorporated herein by reference. Other suitable materials may include ULTANIUM™ (available from Neo-Metrics) and GUM METAL™ (available from Toyota). In some other embodiments, a superelastic alloy, for example a superelastic nitinol can be used to achieve desired properties.

In at least some embodiments, portions or all of catheter 10 may also be doped with, made of, or otherwise include a radiopaque material. Radiopaque materials are understood to be materials capable of producing a relatively bright image on a fluoroscopy screen or another imaging technique during a medical procedure. This relatively bright image aids the user of catheter 10 in determining its location. Some examples of radiopaque materials can include, but are not limited to, gold, platinum, palladium, tantalum, tungsten alloy, polymer material loaded with a radiopaque filler, and the like. Additionally, other radiopaque marker bands and/or coils may also be incorporated into the design of catheter 10 to achieve the same result.

In some embodiments, a degree of Magnetic Resonance Imaging (MRI) compatibility is imparted into catheter 10. For example, portions of catheter 10 may be made of a material that does not substantially distort the image and create substantial artifacts (i.e., gaps in the image). Certain ferromagnetic materials, for example, may not be suitable because they may create artifacts in an MRI image. In some of these and in other embodiments, portions of catheter 10 may also be made from a material that the MRI machine can image. Some materials that exhibit these characteristics include, for example, tungsten, cobalt-chromium-molybdenum alloys (e.g., UNS: R30003 such as ELGILOY®, PHYNOX®, and the like), nickel-cobalt-chromium-molybdenum alloys (e.g., UNS: R30035 such as MP35-N® and the like), nitinol, and the like, and others.

### EXAMPLES

This disclosure may be further clarified by reference to the following Examples, which serve to exemplify some of the embodiments, and not to limit the invention in any way.

### Example 1

An example catheter (e.g., similar to catheter 10) was manufactured that included push member 66. The example catheter along with a control catheter lacking push member 66 were tested using a standard catheter push test. The push test measured the amount of force at which the catheter buckled. The control catheter averaged 505.651 gm/cm in the push test. Conversely, the example catheter with push member 66 averaged 703.097 gm/cm in the push test. These results indicated that the example catheter with push member 66 demonstrated a 39% increase in pushability as compared to the control catheter.

### Example 2

An example catheter was manufactured that included push member 66. The amount of time it took to deflate the balloon on the example catheter along and on a control catheter lacking push member 66 was measured. The deflation time for the control catheter averaged 61.533 seconds. Conversely, the deflation time for the example catheter with push member 66 averaged 60.65 seconds. These results indicated that the example catheter with push member 66 did not impair the deflation time. Accordingly, the inclusion of push member 66 does not appear to present a barrier to inflation/deflation of the balloon.

### Example 3

An example catheter (e.g., similar to catheter 710) was manufactured that included bond member 774. The example catheter along with a control catheter lacking bond member 774 were tested using a standard catheter push test as described above. The example catheter with bond member 774 demonstrated approximately a 15% increase in pushability as compared to the control catheter.

U.S. patent application Ser. No. 13/475,805, filed May 18, 2012 is herein incorporated by reference.

It should be understood that this disclosure is, in many respects, only illustrative. Changes may be made in details, particularly in matters of shape, size, and arrangement of steps without exceeding the scope of the disclosure. This may include, to the extent that it is appropriate, the use of any of the features of one example embodiment being used in other embodiments. The invention's scope is, of course, defined in the language in which the appended claims are expressed.

What is claimed is:

1. A balloon catheter, comprising:
a catheter shaft including a proximal shaft, a midshaft attached to the proximal shaft, and a distal shaft attached to the midshaft;
a balloon coupled to the distal shaft;
wherein an inflation lumen is defined in the catheter shaft that extends from the proximal shaft, through the midshaft, and into the distal shaft, the inflation lumen being in fluid communication with the balloon;
a core wire extending through a portion of the inflation lumen; and
a push member coupled to the core wire, the push member being configured to engage an inner wall surface of the catheter shaft.

2. The balloon catheter of claim 1, wherein the push member includes a bond member that bonds the core wire to the catheter shaft.

3. The balloon catheter of claim 2, wherein the midshaft has a guidewire port formed therein and wherein the bond member is disposed distally of the guidewire port.

4. The balloon catheter of claim 1, wherein the push member includes a solid member attached to the core wire.

5. The balloon catheter of claim 4, wherein the push member has a non-circular cross-sectional shape.

6. The balloon catheter of claim 1, wherein the push member has a lumen defined therein.

7. The balloon catheter of claim 6, wherein the push member has a circular cross-sectional shape.

8. The balloon catheter of claim 6, wherein the push member has a noncircular cross-sectional shape.

9. The balloon catheter of claim 6, wherein the push member has a crescent shaped cross-sectional shape.

10. A balloon catheter, comprising:
a catheter shaft having a proximal shaft portion, a midshaft portion attached to the proximal shaft portion, and a distal shaft portion attached to the midshaft portion;
a balloon coupled to the catheter shaft;
wherein a guidewire port is formed in the midshaft portion, the guidewire port being in fluid communication with a guidewire lumen extending along a portion of the catheter shaft;
wherein an inflation lumen is defined in the catheter shaft, the inflation lumen being in fluid communication with the balloon;
a core wire extending through the inflation lumen; and
a push member attached to the core wire, the push member contacting an inner wall surface of the catheter shaft.

11. The balloon catheter of claim 10, wherein the push member includes bond member that bonds the core wire to the catheter shaft.

12. The balloon catheter of claim 11, wherein the bond member is disposed distally of the guidewire port.

13. The balloon catheter of claim 10, wherein the push member includes a solid member attached to the core wire.

14. The balloon catheter of claim 10, wherein the push member has a lumen defined therein.

15. The balloon catheter of claim 14, wherein the push member has a circular cross-sectional shape.

16. The balloon catheter of claim 14, wherein the push member has a noncircular cross-sectional shape.

17. A method for manufacturing a balloon catheter, the method comprising:
providing a catheter shaft, the catheter shaft including a proximal shaft portion, a midshaft portion attached to the proximal shaft portion, and a distal shaft portion attached to the midshaft portion;
wherein a guidewire port is formed in the midshaft portion, the guidewire port being in fluid communication with a guidewire lumen extending along a portion of the catheter shaft;
wherein an inflation lumen is defined in the catheter shaft that extends from the proximal shaft portion, through the midshaft portion, and into the distal shaft portion;
providing a core wire having a push member attached thereto; and
disposing the core wire within the inflation lumen so that the push member contacts an inner wall surface of the catheter shaft.

18. The method of claim 17, wherein the push member includes a solid member attached to the core wire.

19. The method of claim 17, wherein the push member has a lumen defined therein.

20. The method of claim 19, wherein the push member has a noncircular cross-sectional shape.

21. A balloon catheter, comprising:
a catheter shaft including a proximal shaft, a midshaft attached to the proximal shaft, and a distal shaft attached to the midshaft;
a balloon coupled to the distal shaft;
wherein an inflation lumen is defined in the catheter shaft that extends from the proximal shaft, through the midshaft, and into the distal shaft, the inflation lumen being in fluid communication with the balloon;
a core wire extending through a portion of the inflation lumen;
a push member coupled to the core wire, the push member being configured to engage an inner wall surface of the catheter shaft; and
wherein the push member includes a cylindrical body having an angled end.

22. A balloon catheter, comprising:
a catheter shaft including a proximal shaft, a midshaft attached to the proximal shaft, and a distal shaft attached to the midshaft;
a balloon coupled to the distal shaft;
wherein an inflation lumen is defined in the catheter shaft that extends from the proximal shaft, through the midshaft, and into the distal shaft, the inflation lumen being in fluid communication with the balloon;
a core wire extending through a portion of the inflation lumen; and
wherein the core wire includes a bent push member region that is configured to engage an inner wall surface of the catheter shaft.
